(19) United States
(12) Patent Application Publication
(10) Pub. No.: US 2023/0055551 A1
(43) Pub. Date: Feb. 23, 2023

(54) PHARMACEUTICAL COMPOSITION FOR PREVENTING OR TREATING NONALCOHOLIC STEATOHEPATITIS CONTAINING ASTER KORAIENSIS NAKAI EXTRACT

(71) Applicant: MAY JUNE LIFE & HEALTH, CO., INC., Chungcheongbuk-do (KR)

(72) Inventors: Seok Hoon WOO, Seoul (KR); Moonkyu KANG, Namyangju-si (KR); Chaeyoung BANG, Seoul (KR)

(73) Assignee: MAY JUNE LIFE & HEALTH, CO., INC., Chungcheongbuk-do (KR)

(21) Appl. No.: 17/788,757

(22) PCT Filed: Dec. 24, 2020
(86) PCT No.: PCT/KR2020/019073

§ 371 Date: Jun. 24, 2022

(30) Foreign Application Priority Data
Dec. 24, 2019 (KR) 10-2019-0174499
Dec. 21, 2020 (KR) 10-2020-0180280

(51) Int. Cl. A61K 36/28; A61P 1/16

(52) U.S. Cl. CPC A61K 36/28; A61P 1/16

(57) ABSTRACT

A pharmaceutical composition containing an Aster koraiensis Nakai extract as an active ingredient, of the present invention, inhibits fat accumulation in liver tissue and treats lobular inflammation, and inhibits ballooning degeneration so as to prevent, alleviate or treat nonalcoholic steatohepatitis.

### TECHNICAL FIELD

The present disclosure relates to a pharmaceutical composition, containing a Gymnaster koraiensis extract, for preventing or treating nonalcoholic steatohepatitis.

### BACKGROUND ART

Nonalcoholic fatty liver disease (NAFLD) is the term for a range of conditions caused by fat deposition in the liver, as measured by radiography or biopsy, even without significant alcohol consumption, drug intake resulting in fatty liver, liver diseases attributed to other accompanying factors, or the like, and encompasses simple steatosis and nonalcoholic steatohepatitis (NASH). Simple fatty liver has a comparatively good prognosis, whereas nonalcoholic steatohepatitis occurs in 10-20% of NAFLD patients and shows inflammation accompanied by hepatocellular damage along with fat deposition in the liver, and is a fatal disease wherein hepatic cirrhosis develops in 9-25% of patients, of which 30-40% die from complications of liver disease.

An increasing social cost by nonalcoholic steatohepatitis has driven many researchers to attempt to develop medicines therefor, but no therapeutic agents have been developed to prevent the occurrence or progression of nonalcoholic steatohepatitis so far, and there is no approved medicine for nonalcoholic steatohepatitis worldwide, and there are only off-label drugs that can be selected as the next best solution in consideration of safety and efficacy for patients. In addition, various pathological conditions are involved in nonalcoholic steatohepatitis, and thus in many cases, general antioxidant and anti-inflammatory efficacy alone cannot lead to sufficient therapeutic efficacy in actual animal experiments and clinical trials. Therefore, there is a growing demand for the development of medicines for nonalcoholic steatohepatitis that is safer and can be taken for a long time.

Gymnaster koraiensis is a deciduous perennial grass belonging to the genus Aster in the class Asteraceae. As can be seen from its scientific name (Gymnaster koraiensis or Aster koraiensis Nakai), the plant is a Korean special plant, and its English name is also Korean daisy (Korean starwort), or also called byeolgaemichyi or goryeossukbujaengi in the Korean language. This plant is distributed in damp areas of mountains and fields in the south of Gyeonggi-do, Korea, and especially grows in many colonies in Gangwon-do, Korea. The height is 50-60 cm, and leaves are long extended forward and pointed at the tip. The leaves are 12-19 cm long and about 1.5-3 cm wide, and small saw teeth are present on the edges of the leaves, and the leaves get smaller as they rise upward. Flowers bloom from June to October, and the flowers in the upper portion are light red purple or light purple, and each one flower hangs at the end of a stem or branch. Fruits are formed with withered petals attached in November, and are 4 mm long and 1.3 mm wide, oval in shape, and hairless.

Young sprouts of Gymnaster koraiensis are eaten as seasoned vegetables or by being put in soups, and the roots thereof, called Asteris Radix, are used as a herbal medicine and prescribed for asthma, wind and cold syndromes, cough, asthma, urinary retention, and the like (Lee, Yumi. (2003). Wildflowers in Korea. Another world, 319-323) and are materials that are safe and harmless to the human body.

Korean Patent No. 10-1187032 confirmed that a Gymnaster koraiensis extract has a hepatoprotective effect against liver damage induced by acetaminophen or tert-butyl hydroperoxide or alcoholic liver damage induced by a high concentration of ethanol. In addition, Korean Patent No. 10-2024987 discloses a feature of adding at least one of a Gymnaster koraiensis extract or a Clematis apiifolia extract to a pharmaceutical composition containing a Silene armeria extract for preventing or treating an inflammatory disease. However, neither the prior art documents nor other documents report whether a Gymnaster koraiensis extract exhibits a direct effect on nonalcoholic fatty liver or nonalcoholic steatohepatitis.

The present inventors found that a Gymnaster koraiensis extract inhibits fat accumulation in the liver tissue and complexly acts on the alleviation of lobular inflammation, thereby exhibiting an effect on the prevention or treatment of nonalcoholic steatohepatitis, and thus completed the present disclosure.

### DISCLOSURE OF INVENTION

### Technical Problem

An aspect of the present disclosure is to provide a pharmaceutical composition, containing a Gymnaster koraiensis extract, for preventing or treating nonalcoholic steatohepatitis.

Another aspect of the present disclosure is to provide a health functional food composition, containing a Gymnaster koraiensis extract, for preventing or alleviating nonalcoholic steatohepatitis.

### Solution to Problem

In an accordance with an aspect of the present disclosure, there is provided a pharmaceutical composition, containing a Gymnaster koraiensis extract as an active ingredient, for preventing or treating nonalcoholic steatohepatitis.

In the pharmaceutical composition, the Gymnaster koraiensis extract may be obtained by extraction with at least one solvent selected from the group consisting of water, a C1-C4 lower alcohol, acetone, ethyl acetate, and hexane.

The C1-C4 lower alcohol may be methanol or ethanol.

The Gymnaster koraiensis extract may be obtained by extraction from an aerial part including a flower of Gymnaster koraiensis or extraction from a flower of Gymnaster koraiensis.

In accordance with another aspect of the present disclosure, there is provided a health functional food composition, containing a Gymnaster koraiensis extract as an active ingredient, for preventing or alleviating nonalcoholic steatohepatitis.

In the health functional food composition, the Gymnaster koraiensis extract may be obtained by extraction with at least one solvent selected from the group consisting of water, a C1-C4 lower alcohol, acetone, ethyl acetate, and hexane.

The C1-C4 lower alcohol may be methanol or ethanol.

The Gymnaster koraiensis extract may be obtained by extraction from an aerial part including a flower of Gymnaster koraiensis or extraction from a flower of Gymnaster koraiensis.

Hereinafter, the present disclosure will be described in more detail.

The present disclosure is directed to a pharmaceutical composition, containing a Gymnaster koraiensis extract as an active ingredient, for preventing or treating nonalcoholic steatohepatitis.

The whole plant of Gymnaster koraiensis may be used, and the aerial part including young sprouts, leaves, stems, branches, flowers, and fruits, or roots thereof, as portions of the plant, may be used. Although not limited, the Gymnaster koraiensis extract of the present disclosure is preferably an extract obtained by extraction of the aerial part including flowers of Gymnaster koraiensis or an extract obtained by extraction of flowers of Gymnaster koraiensis, and more preferably an extract obtained by extraction of flowers of Gymnaster koraiensis. The Gymnaster koraiensis flower extract was three times better in terms of an inflammation inhibitory effect in hepatic lobular cells, compared with extracts of the aerial part excluding flowers, for example, leaves, stems, and branches of Gymnaster koraiensis. As used herein, the term “aerial part including flowers of Gymnaster koraiensis” refers to “part including all of flowers, leaves, stems, and branches of Gymnaster koraiensis”.

The Gymnaster koraiensis extract is preferably an extract obtained by extraction with at least one solvent selected from the group consisting of water, a C1-C4 lower alcohol, acetone, ethyl acetate, and hexane. The C1-C4 lower alcohol may be methanol, ethanol, propanol, isopropanol, butanol, or the like. The Gymnaster koraiensis extract is more preferably an extract obtained by extraction with methanol or ethanol.

The solvent may be added at a weight of 2 to 100 times, preferably 2 to 50 times, and more preferably 5 to 30 times the dry weight of Gymnaster koraiensis, for extraction.

The Gymnaster koraiensis extract may be extracted by a common extraction method in the art, for example, a method using an extraction apparatus for hot water extraction, immersion extraction, supercritical extraction, subcritical extraction, high temperature extraction, high pressure extraction, reflux cooling extraction, or ultrasonic extraction, or a method of using an adsorption resin containing XAD and HP-20. Reflux extraction at an elevated temperature or extraction at room temperature is preferable, but is not limited thereto. The number of times of extraction of the Gymnaster koraiensis extract is preferably 1 to 5, and three times of repeated extraction are more preferable, but are not limited thereto. One to five times of extraction may be performed by ultrasonic extraction. The temperature of extraction is preferably 10° C. to 100° C., and extraction at 50° C. to 100° C. is more preferable, but is not limited thereto. The time of extraction for each time is preferably 1 to 12 hours, and more preferably 2 to 8 hours, but is not limited thereto.

According to a specific embodiment of the present disclosure, a method for preparing a Gymnaster koraiensis extract for prevention or treatment of nonalcoholic steatohepatitis comprises the steps of: 1) obtaining a dry product obtained by drying Gymnaster koraiensis; 2) adding ethanol to the dry product, followed by reflux extraction and then filtration, thereby obtaining an ethanol extract; and 3) concentrating the ethanol extract under reduced pressure to obtain a Gymnaster koraiensis extract. In addition, a powder form of Gymnaster koraiensis extract may be obtained through a drying step, after the concentration under reduced pressure.

As for an animal test model with nonalcoholic steatohepatitis induced by ad-lib intake of a high-fatty diet, the oral administration of a Gymnaster koraiensis extract obtained by the preparation method was identified to have effects of inhibiting fat accumulation in the liver tissue, which corresponds to histopathological characteristic of nonalcoholic steatohepatitis, as well as treating lobular inflammation and inhibiting hepatocellular ballooning. Additionally, the extract significantly reduced indicators, such as AST and ALT levels, as a result of hematological evaluation.

Therefore, the Gymnaster koraiensis extract of the present disclosure can be advantageously used as a pharmaceutical composition for preventing or treating nonalcoholic steatohepatitis.

The pharmaceutical composition of the present disclosure may be formulated in the form of: an oral formulation, such as a powder, granules, a tablet, a capsule, a suspension, an emulsion, a syrup, an aerosol, or a jelly; an externally applied preparation; a suppository; and a sterile injectable solution, according to ordinary methods. Examples of a carrier, an excipient, and a diluent that may be contained in the composition of the present disclosure may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and a mineral oil.

The composition, when formulated as a preparation, may be formulated using a diluent or an excipient, such as a filler, an extender, a binder, a wetting agent, a disintegrant, or a surfactant, which are commonly used. Exemplary solid preparations for oral administration include a tablet, a pill, a powder, granules, a capsule, and the like, and such solid preparations may be prepared by mixing the Gymnaster koraiensis extract with at least one excipient, for example, starch, calcium carbonate, sucrose or lactose, gelatin, or the like. Also, a lubricant, such as magnesium stearate or talc, may be used in addition to simple excipients. Exemplary liquid preparations for oral administration include a suspension, an oral solution, an emulsion, a syrup, and the like. In addition to simple diluents, such as water, liquid, and paraffin, several excipients, for example, a wetting agent, a sweetener, an aroma, and a preservative, may be contained therein. Exemplary preparations for parenteral administration include a sterile aqueous solution, a non-aqueous solvent, a suspension, an emulsion, a freeze-dried preparation, and a suppository. Examples of the non-aqueous solvent and suspension may include propylene glycol, polyethylene glycol, a vegetable oil such as olive oil, an injectable ester such as ethylolate, and the like. A base material for the suppository may include Witepsol, Macrogol, Tween 61, cocoa butter, laurin butter, glycerogelatin, and the like.

The composition of the present disclosure may be administered orally or parenterally, and any parenteral administration method may be used. In addition, systemic or topical administration is possible, but systemic administration is more preferable, and intravenous administration is most preferable.

A preferable dose of the composition of the present disclosure may vary depend on the condition and weight of a patient, severity of a disease, form of a drug, and route and period of administration, but may be selected as appropriate by a person skilled in the art. However, for desirable effects, the composition of the present disclosure is preferably administered at 0.0001 to 1 g/kg, preferably 0.001 to 200 mg/kg per day. The administration may be performed once or several times in divided doses per day. The dose is not in any way intended to limit the scope of the present disclosure.

In accordance with another aspect of the present disclosure, there is provided a health functional food composition, containing a Gymnaster koraiensis extract as an active ingredient, for preventing or alleviating nonalcoholic steatohepatitis.

The type of health functional food is not particularly limited. Examples of the food to which the material may be added include a drink, a meat, a sausage, a bread, a biscuit, a rice cake, a chocolate, a candy, a snack, a cookie, a pizza, an instant noodle, other noodles, a gum, a dairy product including ice cream, a soup, a beverage, an alcohol beverage, a vitamin complex, a milk product, and a processed milk product, and include all health functional foods in a common sense.

The Gymnaster koraiensis extract of the present disclosure may be added as it is to a food or may be used with other foods or food ingredients, and may be appropriately used according to common methods. The mixing amount of an active ingredient may be suitably determined according to the purpose of use (prevention or alleviation) thereof. The amount of the extract in the health functional food may be generally added in an amount of 0.1 to 90 parts by weight of the total weight of the food. However, in the case of long-term intake for the purpose of health and hygiene or for the purpose of controlling health, the amount may be equal to or less than the above range, and the active ingredient may be used in an amount equal to or more than the above range due to no problem in terms of safety.

Providing that the health functional beverage composition of present disclosure contains the extract as an essential ingredient at the indicated proportions, there is no particular limitation on the other ingredients, so the composition, like conventional beverage, may contain various flavoring agents, natural carbohydrates, and the like, as additional ingredients. Examples of the foregoing natural carbohydrates may include ordinary sugars, such as monosaccharides (e.g., glucose, fructose, etc.), disaccharides (e.g., maltose, sucrose, etc.), and polysaccharides (e.g., dextrin, cyclodextrin, etc.); and sugar alcohols, such as xylitol, sorbitol, and erythritol. As flavoring agents except for the foregoing flavoring agents, natural flavoring agents (thaumatin, and stevia extract (e.g., rebaudioside A, glycyrrhizin, etc.)) and synthetic flavoring agents (saccharin, aspartame, etc.) may be advantageously used. The proportion of the natural carbohydrate relative to 100 ml of the composition of the present disclosure is generally about 1 to 20 g, and preferably 5 to 12 g.

In addition to the above ingredients, the composition containing the Gymnaster koraiensis extract of the present disclosure may contain several types of nutrients, vitamins, minerals (electrolytes), flavoring agents such as synthetic flavoring agents and natural flavoring agents, coloring agents, extenders (cheese, chocolate, etc.), pectic acid and salts thereof, alginic acid and salts thereof, organic acids, protective colloid thickeners, pH adjusters, stabilizers, preservatives, glycerin, alcohol, carbonating agents used for carbonated drinks, and the like. Additionally, the composition containing the Gymnaster koraiensis extract of the present disclosure may contain fruit flesh for manufacturing natural fruit juice, fruit juice drinks, and vegetable drinks. These ingredients may be used either alone or in combination. The proportion of such an additive is not greatly important, but is usually selected within a range of 0.1 to about 20 parts by weight relative to 100 parts by weight of the Gymnaster koraiensis extract of the present disclosure or a fraction thereof.

### Advantageous Effects of Invention

The present disclosure, which is directed to a pharmaceutical composition containing a Gymnaster koraiensis extract as an active ingredient, inhibits fat accumulation in the liver tissue, treats lobular inflammation, and inhibits ballooning, and thus can prevent, alleviate, or treat nonalcoholic steatohepatitis.

### BRIEF DESCRIPTION OF DRAWINGS

FIG. 1 is a diagram showing nonalcoholic fatty liver disease (NAFLD) activity scores of a normal group (normal), a control group (control), Example 1 (MJAK) administration groups, an Example 2 (MJAK-F) administration group, an Example 3 (MJAK-L) administration group, an Example 4 (MJAK-S) administration group, and positive control groups (silymarin and pioglitazone) in a high-fat diet fed model.

FIG. 2 is a diagram showing steatosis assessment results of a normal group, a control group, Example 1 to Example 4 administration groups, and positive control groups in a high-fat diet fed model.

FIG. 3 is a diagram showing lobular inflammation assessment results of a normal group, a control group, Example 1 to Example 4 administration groups, and positive control groups in a high-fat diet fed model.

FIG. 4 is a diagram showing ballooning assessment results of a normal group, a control group, Example 1 to Example 4 administration groups, and positive control groups in a high-fat diet fed model.

FIG. 5 shows Oil-red O staining results of mouse liver tissues of a normal group, a control group, Example 1 administration groups, and a positive control group in a high-fat diet fed model.

FIG. 6 shows H&E staining results of mouse liver tissues of a normal group, a control group, Example 1 administration groups, and a positive control group in a high-fat diet fed model.

FIG. 7 presents diagrams showing AST and ALT levels of a normal group, a control group, Example 1 administration groups, and a positive control group in a high-fat diet fed model.

### BEST MODE FOR CARRYING OUT THE INVENTION

Hereinafter, preferable exemplary embodiments of the present disclosure will be described in detail. However, the present disclosure is not limited to the exemplary embodiments described herein, and thus may be embodied into different forms. These exemplary embodiments are provided so that the present disclosure will be thorough and complete and will fully convey the scope of the disclosure to those skilled in the art.

### TEST EXAMPLE 1

### Preparation of Gymnaster koraiensis extract

1. Preparation of Gymnaster koraiensis Extract of Example 1

The aerial part including flowers of Gymnaster koraiensis was prepared by collection in August 2018 in Asan-si, Chungcheongnam-do, Korea. Gymnaster koraiensis, which had been cleanly washed with water and then dried to have a moisture content of 9.2% or less, was finely cut, and then 20 L of 95% ethanol was added to 1 kg thereof, followed by reflux extraction at 70° C. for 4 hours, and thereafter, the extract was filtered. To the residue remaining after the filtration of the extract was again added 20 L of 95% ethanol, and reflux extraction at 70° C. for 4 hours was further repeated two times, thereby obtaining 60 L of a total extract. The 60 L of extract was concentrated under reduced pressure at 45-55° C. to obtain 168.4 g of a 95% ethanol extract, and this extract was called a Gymnaster koraiensis extract of Example 1 (or called “MJAK”).

2. Preparation of Gymnaster koraiensis extracts of Examples 2 to 4

The aerial part including flowers of Gymnaster koraiensis collected as in Example 1 was separated into a flower part of Gymnaster koraiensis and a part of leaves, stems, and branches of Gymnaster koraiensis, which were the other part of the aerial part not including flowers of Gymnaster koraiensis. These parts were cleanly washed with water, dried to have a moisture content of 9.2% or less, and then finely cut, thereby preparing 1 kg for each part. Extraction was performed by the same method as in Example 1 to obtain 32.3 g of a flower extract of Gymnaster koraiensis, assigned as Example 2 (or “MJAK-F”), and 170.2 g of an extract of leaves, stems, and branches of Gymnaster koraiensis, assigned as Example 3 (or “MJAK-L”).

In addition, young sprouts collected in April 2018 in Asan-si, Chungcheongnam-do, Korea, were cleanly washed with water, dried to have a moisture content of 9.2% or less, and then finely cut, thereby preparing 1 kg. Extraction was performed by the same method as in Example 1 to obtain 121.6 g of a young sprout extract of Gymnaster koraiensis, assigned as Example 4 (or “MJAK-S”).

### TEST EXAMPLE 2

### Efficacy Assessment in High-Fat Diet (HFD) Fed Nonalcoholic Steatohepatitis Model

1. Test Method

For the construction of a nonalcoholic fatty liver animal model, 5-week-old male C57/BL6J mice (Charles River Laboratories Japan Inc., Japan) were acclimatized for one week in an animal test facility where a temperature of 22-25° C., a relative humidity of 50-60%, a light-dark cycle of 12 hours, and an illuminance of 200-300 LUX were maintained. Two mice were housed in each polycarbonate box for mice with regular feed (Daehan Biolink Co., Ltd., Republic of Korea) and drinking water ad libitum during the acclimatization period of one week.

After the acclimatization, eight animals per group were grouped as follows such that the average body weights were similar. A normal group was fed a normal diet (Rodent Diet with 10% kcal fat, Republic of Korea), and a control group, groups administered Gymnaster koraiensis extracts of Examples 1 to 4, and a silymarin or pioglitazone administration group as a positive control group, were fed a high-fat diet (60% kcal high fat diet, Research Diets Inc. NJ, USA) ad libitum for 12 weeks.

TABLE 1
Test group configuration and condition in high-fat diet fed nonalcoholic steatohepatitis model

| Test group | | | Condition |
|---|---|---|---|
| G1 | Normal group | | Normal diet + 0.5% CMC |
| G2 | Control group | | High-fat diet + 0.5% CMC |
| G3 | Example 1 (MJAK) low | | High-fat diet + 0.5% CMC + MJAK 125 mg/kg |
| G4 | Example 1 (MJAK) medium | | High-fat diet + 0.5% CMC + MJAK 250 mg/kg |
| G5 | Example 1 (MJAK) high | | High-fat diet + 0.5% CMC + MJAK 500 mg/kg |
| G6 | Example 2 (MJAK-F) medium | | High-fat diet + 0.5% CMC + MJAK 250 mg/kg |
| G7 | Example 3 (MJAK-L) medium | | High-fat diet + 0.5% CMC + MJAK 250 mg/kg |
| G8 | Example 4 (MJAK-S) medium | | High-fat diet + 0.5% CMC + MJAK 250 mg/kg |
| G9 | Positive control | Silymarin | High-fat diet + 0.5% CMC + silymarin 200 mg/kg |
| G10 | | Pioglitazone | High-fat diet + 0.5% CMC + pioglitazone 10 mg/kg |

The doses of silymarin and pioglitazone as positive control groups were set to 200 mg/kg and 10 mg/kg, respectively, with reference to the reported toxicity and side effects thereof. The normal group was orally administered a 0.5% carboxymethylcellulose (CMC) solution once a day in the morning for 84 days, a total of 84 times, from the day of feeding of the normal diet. The control group was orally administered a 0.5% CMC solution once a day in the morning for 84 days, a total of 84 times, from the day of feeding of the high-fat diet. The Examples 1 to 4 administration groups and the positive control groups were orally administered Example 1 (125, 250, and 500 mg/kg), Examples 2 to 4 (250 mg/kg), silymarin 200 mg/kg, and pioglitazone 10 mg/kg, respectively, which were added to a 0.5% CMC solution, once a day in the morning for 84 days, a total of 84 times, from the day of feeding of the high-fat diet.

2. Histopathological Assessment Methods and Results

(1) Preparation of H&E Staining Slides and Oil-Red O Staining Slides

For histopathological examination, the mice were sacrificed and the liver tissue was resected to prepare hematoxylin-eosin (H&E) stained slides and Oil-red O staining slides. H&E staining is a staining method that can broadly identify the whole sample and is performed to observe a change in cell size due to adipogenesis in the liver tissue and fat globules generated among cells. Oil-red O staining is performed to determine the amount of fat produced in the liver tissue.

The resected liver tissue of mice was fixed in a 4% paraformaldehyde solution at 4° C. for 3 days, transferred into a 30% sucrose solution, and stored at 4° C. before Oil-red O staining. The tissue for hematoxylin & eosin (H&E) staining was immersed and fixed in 10% formalin.

First, the tissue for Oil-red-0 staining was cryo-sectioned into a thickness of 10 μm, attached to tissue slides, rehydrated, subjected to Oil-red-O staining, dehydrated, and then sealed.

The tissue for H&E staining fixed in 10% formalin was sectioned into a thickness of 5 μm, attached to tissue slides, stained with a hematoxylin solution and an eosin solution in that order, dehydrated, and then sealed.

(2) Histopathological Assessment Results

The H&E-stained slides were histopathologically assessed under a microscope, and the nonalcoholic fatty liver disease (NAFLD) activity score (NAS) based on the criteria on Table 2 was numerically recorded. The nonalcoholic fatty liver disease activity score is expressed as a sum of assessment scores for changes in 1) steatosis, 2) lesions such as lobular inflammation, and 3) hepatocellular ballooning, and is widely used for evaluating the severity of nonalcoholic steatohepatitis.

TABLE 2
NAFLD activity score (NAS) assessment criteria

| Item | Definition | Score |
|---|---|---|
| Steatosis | Low- to medium-power evaluation | |
| | of parenchymal involvement by | |
| | steatosis | |
| | <5% | 0 |
| | 5~33% | 1 |
| | >33~66% | 2 |
| | >66% | 3 |
| Lobular | Overall assessment of all | |
| | inflammatory foci | |
| Inflammation | No foci | 0 |
| | <2 foci per 200x field | 1 |
| | 2-4 foci per 200x field | 2 |
| | >4 foci per 200x field | 3 |
| Ballooning | None | 0 |
| | Few balloon cells | 1 |
| | Many cells/prominent ballooning | 2 |

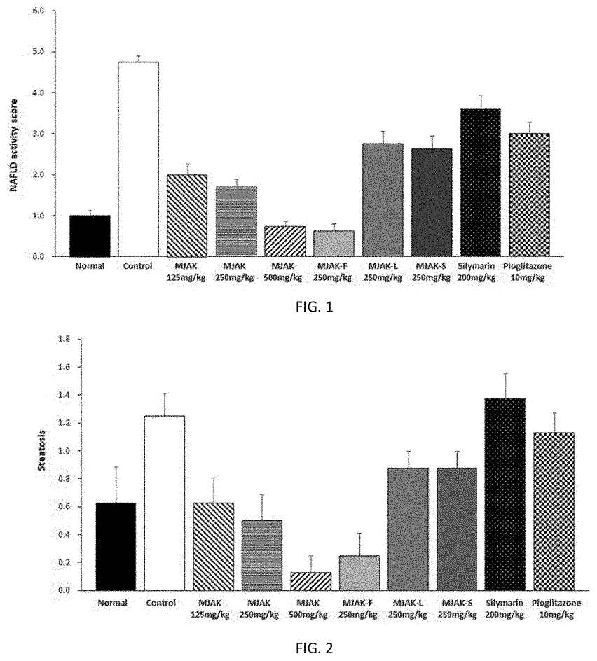

As shown in FIG. 1, the groups administered the Gymnaster koraiensis extract (MJAK) of Example 1 were identified to show a significant decrease in NAFLD activity score in a dose-dependent manner compared with the control group or the silymarin or pioglitazone administration group. The Example 1 (500 mg/kg) administration group was identified to have a NAFLD activity score equal to or lower than that of the normal group.

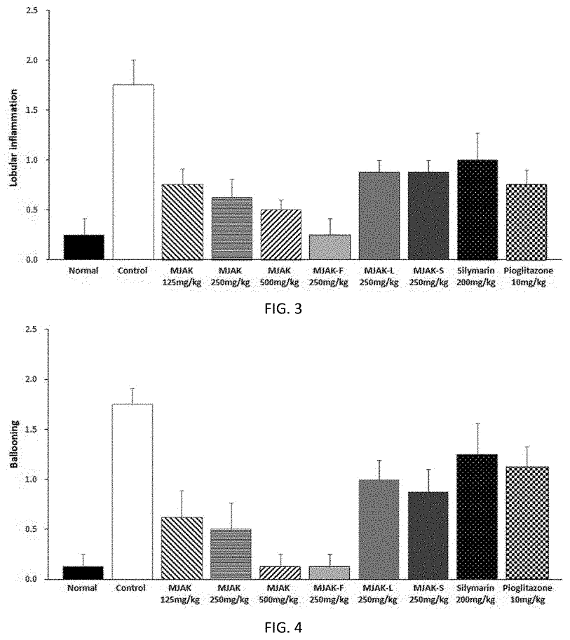

As shown in FIG. 2, the groups administered the Gymnaster koraiensis extract of Example 1 showed a significantly excellent effect of inhibiting steatosis in the liver tissue compared with the control group, the silymarin group, and the pioglitazone group. Furthermore, the Example 1 administration groups were identified to have excellent effects of mitigating lobular inflammatory cell infiltration and inhibiting ballooning in a dose-dependent manner compared with the silymarin or pioglitazone administration group (FIGS. 3 and 4).

As for the effects of the extracts of different parts of Gymnaster koraiensis, the groups administered the extract of the aerial part including flowers of Example 1 (MJAK), the extract of flowers of Example 2 (MJAK-F), the extract of leaves, stems, and branches of Example 3 (MJAK-L), and the extract of young sprouts of Example 4 (MJAK-S) at 250 mg/kg were identified to show a decrease in NAFLD activity score and have effects of inhibiting steatosis in the liver tissue, lobular inflammation, and ballooning, compared with the control group. In particular, the extract of the aerial part including flowers of Gymnaster koraiensis of Example 1 or the extract of flowers of Gymnaster koraiensis of Example 2 was identified to show a significant decrease in NAFLD activity score and be at three times excellent in terms of the effects of inhibiting steatosis in the liver tissue, ballooning, and lobular inflammation, compared with the extract of leaves, stems, and branches of Gymnaster koraiensis of Example 3 or the extract of young sprouts of Gymnaster koraiensis of Example 4 (see FIGS. 1 to 4).

FIG. 5 shows the results of observing the Oil-red O stained slides. It was identified that large fat globules were distributed throughout the liver tissue of the control group, but it was observed that the size of fat globules was decreased in a concentration-dependent manner by administration of Example 1 at different concentrations, and the liver tissue by the example at 500 mg/kg was the same as that of the normal group.

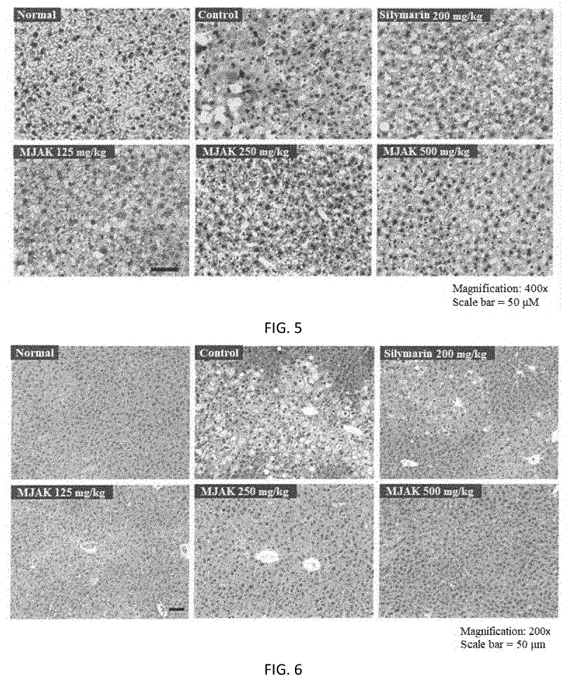

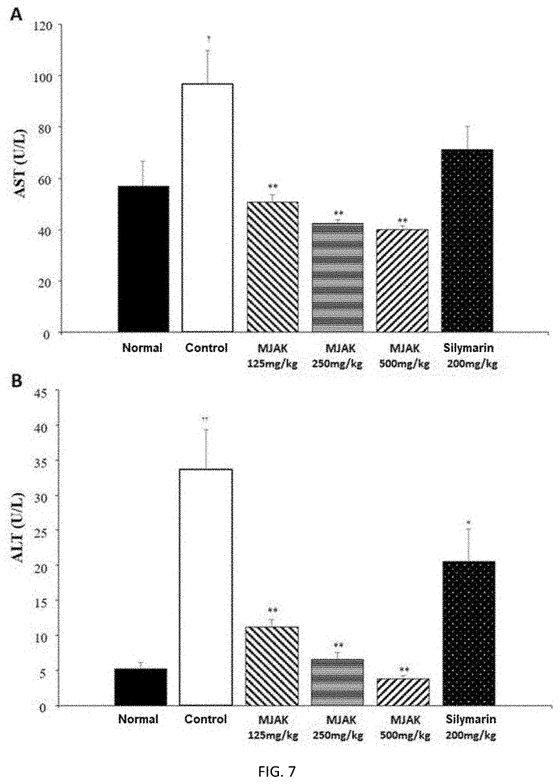

FIG. 6 shows the results of observing the H&E stained slides. The control group was observed to have an increase in size of liver tissue cells and adipogenesis thereamong, but it was identified that cell hypertrophy disappeared and the amount of fat produced among cells was significantly decreased, by administration of Example 1 at different concentrations.

It was therefore identified that the present disclosure was effective in the prevention or treatment of nonalcoholic steatohepatitis.

3. Clinicopathological Assessment Results

(1) AST and ALT Analysis Methods

After completion of the high-fat diet feeding test for C57/BL6J mice, the mice were fasted for 12 hours before sacrifice, and then blood was collected from the abdominal aorta of C57/BL6J mice, followed by centrifugation to separate serum, which was then used for aspartate aminotransferase (AST) and alanine aminotransferase (ALT) analyses.

(2) AST and ALT Analysis Results

AST and ALT in FIG. 7 are blood indicators that indicating liver functions and, especially, used as indicators of liver cytotoxicity. The control group induced with nonalcoholic fatty liver showed an increase in AST by about 1.7 times and an increase in ALT by about 6.7 times compared with the normal group, whereas the increases in AST and ALT levels were significantly suppressed in a concentration-dependent manner by the administration of Example 1 (MJAK) at different concentrations.

It was identified through the efficacy assessments in the nonalcoholic steatohepatitis animal model that the Gymnaster koraiensis extracts had excellent effects on the prevention, alleviation, and treatment of nonalcoholic steatohepatitis.

1. A pharmaceutical composition, containing a Gymnaster koraiensis extract as an active ingredient, for preventing or treating nonalcoholic steatohepatitis.

2. The pharmaceutical composition of claim 1, wherein the Gymnaster koraiensis extract is obtained by extraction with at least one solvent selected from the group consisting of water, a C1-C4 lower alcohol, acetone, ethyl acetate, and hexane.

3. The pharmaceutical composition of claim 2, wherein the C1-C4 lower alcohol is methanol or ethanol.

4. The pharmaceutical composition of claim 1, wherein the Gymnaster koraiensis extract is obtained by extraction from an aerial part including a flower of Gymnaster koraiensis or extraction from a flower of Gymnaster koraiensis.

5. A health functional food composition, containing a Gymnaster koraiensis extract as an active ingredient, for preventing or alleviating nonalcoholic steatohepatitis.

6. The health functional food composition of claim 5, wherein the Gymnaster koraiensis extract is obtained by extraction with at least one solvent selected from the group consisting of water, a C1-C4 lower alcohol, acetone, ethyl acetate, and hexane.

7. The health functional food composition of claim 6, wherein the C1-C4 lower alcohol is methanol or ethanol.

8. The health functional food composition of claim 5, wherein the Gymnaster koraiensis extract is obtained by extraction from an aerial part including a flower of Gymnaster koraiensis or extraction from a flower of Gymnaster koraiensis.

9. A method for preventing or treating nonalcoholic steatohepatitis, comprising administering to in a subject in need thereof an effective amount of a Gymnaster koraiensis extract.

10. The method of claim 9, wherein the Gymnaster koraiensis extract is obtained by extraction with at least one solvent selected from the group consisting of water, a C1-C4 lower alcohol, acetone, ethyl acetate, and hexane.

11. The method of claim 10, wherein the C1-C4 lower alcohol is methanol or ethanol.

12. The method of claim 9, wherein the Gymnaster koraiensis extract is obtained by extraction from an aerial part including a flower of Gymnaster koraiensis or extraction from a flower of Gymnaster koraiensis.
